(19) United States
(12) Patent Application Publication
(10) Pub. No.: US 2020/0345500 A1
(43) Pub. Date: Nov. 5, 2020

(54) TISSUE ENGINEERING MENISCAL COMPOSITE SCAFFOLD AND PREPARATION METHOD THEREOF

(71) Applicant: CHINESE PLA GENERAL HOSPITAL, Beijing (CN)

(72) Inventors: Quanyi GUO, Beijing (CN); Weimin GUO, Beijjing (CN); Shibi LU, Beijing (CN); Shuyun LIU, Beijing (CN); Xiang SUI, Beijing (CN); Jingxiang HUANG, Beijing (CN); Mingxue CHEN, Beijing (CN); Zhenyong WANG, Beijing (CN); Shuang GAO, Beijing (CN); Zhiguo YUAN, Beijing (CN)

(21) Appl. No.: 16/765,161

(22) PCT Filed: May 16, 2019
(86) PCT No.: PCT/CN2019/087228

§ 371 Date: May 18, 2020

(30) Foreign Application Priority Data
Aug. 30, 2018 (CN) 201811003747.7

(51) Int. Cl. A61F 2/30; A61L 27/56; A61L 27/26; A61L 27/36; A61L 27/48; B33Y 10/00; B33Y 50/02; B33Y 80/00; B33Y 40/20; B33Y 70/10

(52) U.S. Cl. CPC A61F 2/30756; A61F 2002/30985; A61L 27/56; A61L 27/26; A61L 27/3633; A61L 27/48; A61L 27/3654; A61F 2/30942; B33Y 10/00; B33Y 50/02; B33Y 80/00; B33Y 40/20; B33Y 70/10; A61F 2002/30766; A61F 2002/30131; A61F 2002/30062; A61F 2002/30784; A61L 2430/06; A61F 2002/30952; A61F 2/30771

(57) ABSTRACT

A tissue engineering meniscal composite scaffold (100) and a preparation method thereof. The meniscal composite scaffold (100) includes: a scaffold (110), wherein the scaffold (110) is C-shaped and has a shape which is consistent with the original shape of the meniscus to be regenerated, the scaffold (110) comprises a plurality of first degradable polymer fibers (111) extending along the circumferential direction of the scaffold (110) and a plurality of second degradable polymer fibers (112) extending along the radial direction of the scaffold (110), and the first degradable polymer fibers (111) form a multilayer intersection with the second degradable polymer fibers (112) and thereby generating a frame structure having a plurality of first apertures; and a matrix material (120) composited inside the plurality of first apertures of the scaffold (110) to form a meniscal composite scaffold (100) having a plurality of second apertures.

### CROSS-REFERENCE TO RELATED APPLICATION

This application is a National Stage of International Application No. PCT/CN2019/087228, filed on May 16, 2019, which claims priority to Chinese Patent Application No. 201811003747.7, filed on Aug. 30, 2018, both of which are hereby incorporated by reference in their entireties.

### TECHNOLOGY FIELD

The present invention is related to the field of medical devices, and particularly to a tissue engineering meniscal composite scaffold and a preparation method thereof.

### BACKGROUND

Menisci are located between the femoral condyle and the tibial plateau, one inside and the other outside. Their main function is to nourish, lubricate and stabilize the knee joint, and to cushion the knee joint stress. Damage and degeneration of meniscus will cause dysfunction of meniscus, the cartilage of knee will be less protected, which will lead to knee disorders. Damage and degeneration of meniscus can be dealt with clinically by total or partial meniscectomy, this offers short-term relief of knee disorders. However, if the damage and degeneration of meniscus happens in the avascular inner portion, self-healing is often difficult after the meniscectomy, this will inevitably cause long-term degenerative changes of the joint, and lead to knee osteoarthritis.

The development of tissue engineering and regenerative medicine has provided new therapies for meniscus repair. Among them, tissue engineering scaffold, a carrier for seed cells and active substances such as biological signal molecules, plays a vital role in the regeneration of new tissues. However, nowadays it is still difficult to provide a tissue engineering scaffold which is well balanced between good mechanical properties and good biocompatibility, and the shape, structure, mechanical properties and physiological function of the newly formed meniscus still have many shortcomings, which may even change the knee microenvironment and accelerate degenerative joint changes or exacerbate knee osteoarthritis.

### SUMMARY

To solve the above problems, the present application provides a tissue engineering meniscal composite scaffold and a preparation method thereof, this meniscal composite scaffold is well balanced between good mechanical properties and good biocompatibility, provides excellent microenvironment desired for cell growth, and the newly formed meniscus has good shape, structure, mechanical properties and physiological function.

In the first aspect, the present application provides a tissue engineering meniscal composite scaffold including:

a scaffold, which is C-shaped and has a shape which is consistent with the original shape of the meniscus to be regenerated, comprising a plurality of first degradable polymer fibers extending along the circumferential direction of the scaffold and a plurality of second degradable polymer fibers extending along the radial direction of the scaffold; the first degradable polymer fibers form a multilayer intersection with the second degradable polymer fibers and thereby generating a frame structure having a plurality of first apertures, the diameter of the first apertures is 750 μm-1500 μm;

matrix material, which is composited inside the plurality of first apertures to form a meniscal composite scaffold having a plurality of second apertures, the diameter of the second apertures is 90 μm-150 μm.

In the second aspect, the present application provides a method for preparing a tissue engineering meniscal composite scaffold comprising following steps:

a modeling step to generate a three-dimensional data model of the undamaged meniscus to be regenerated before damage;

a printing step, in which a scaffold is printed according to the three-dimensional data model using degradable polymer as raw material, and the scaffold is C-shaped and has a shape which is consistent with the original shape of the meniscus to be regenerated, the scaffold comprises a plurality of first degradable polymer fibers extending along the circumferential direction of the scaffold and a plurality of second degradable polymer fibers extending along the radial direction of the scaffold; the first degradable polymer fibers form a multilayer intersection with the second degradable polymer fibers and thereby generating a frame structure having a plurality of first apertures, the diameter of the first apertures is 750 μm-1500 μm;

a hydrophilic treatment step, in which the scaffold is subjected to hydrophilic treatment;

a preparation step for preparing lyophilized meniscal composite scaffold, in which a solution comprising matrix material is filled into the plurality of first apertures of the scaffold and the scaffold is subjected to lyophilization to obtain the lyophilized meniscal composite scaffold;

a post-processing step, in which the lyophilized meniscal composite scaffold is subjected to cross-linking treatment and sterilization treatment to obtain a meniscal composite scaffold having a plurality of second apertures, the diameter of the second apertures is 90 μm-150 μm.

The embodiments of the present application may have the following advantages.

The tissue engineering meniscal composite scaffold has a configuration which fits an individual precisely, is well balanced between good mechanical properties and good biocompatibility, is able to provide excellent microenvironment desired for cell growth, is favorable for the growth, proliferation and re-differentiation of the cells under both in vivo and in vitro conditions, and therefore is able to facilitate the regeneration of the damaged meniscus in the avascular inner portion, and so that the newly formed meniscus has good shape, structure, mechanical properties and physiological function to protect the knee joint.

### BRIEF DESCRIPTION OF THE DRAWINGS

To further illustrate the technical solutions of the embodiments of the present application, the drawings used in the embodiments of the present application will be briefly described below. The drawings described below apparently only show some embodiments of the present application. For those of ordinary skill in the art, other embodiments can be obtained based on the drawings without the exercise of inventive skills.

FIG. 1A-FIG. 1B are schematic drawings of the tissue engineering meniscal composite scaffold of an embodiment of the present application.

FIG. 2 shows the scanning electron microscope cross section graph of the tissue engineering meniscal composite scaffold of an embodiment of the present application.

FIG. 3 is a schematic drawing of the scaffold of the tissue engineering meniscal composite scaffold of an embodiment of the present application.

FIG. 4 is a schematic drawing showing the intersected first degradable polymer fibers and second degradable polymer fibers in the scaffold of an embodiment of the present application.

FIG. 5A-FIG. 5D show medical images from different angels of a sheep medial meniscus in an embodiment of the present application.

FIG. 6 shows the newly formed meniscal tissue after the tissue engineering meniscal composite scaffold of Example 4 of the present application is implanted into the damaged medial meniscus in the knee of a sheep.

### DETAILED DESCRIPTION

The present application is further illustrated by the following particular embodiments to clarify the objectives, the technical solutions and the beneficial technical effects of the present application. It should be understood that the embodiments described in this specification are only for explaining the application, rather than limiting the scope of the present application in any way.

For simplicity, only some of the ranges are explicitly stated. However, any lower limit may be combined with any upper limit to disclose a range that is not explicitly stated; and any lower limit may be combined with any other lower limit to disclose a range that is not explicitly stated, and likewise any upper limit may be combined with any other upper limit to disclose a range that is not explicitly stated. In addition, although not explicitly stated, every point or individual value between the endpoints of a range is included in the range. Therefore, each point or individual value itself can be used as lower limit or upper limit to be combined with any other point or individual value or with other lower limit or upper limit to disclose a range that is not explicitly stated.

Unless otherwise indicated, the term “more” in the phrase “one or more” means two or more.

The summary of the present application is not intended to describe every embodiment or every implementation of this application. Illustrative embodiments are described below in more detail. Guidance is provided through a series of embodiments throughout the present application, any of these embodiments can be combined. In each listing, only representatives are stated, this should not be interpreted as exhaustive.

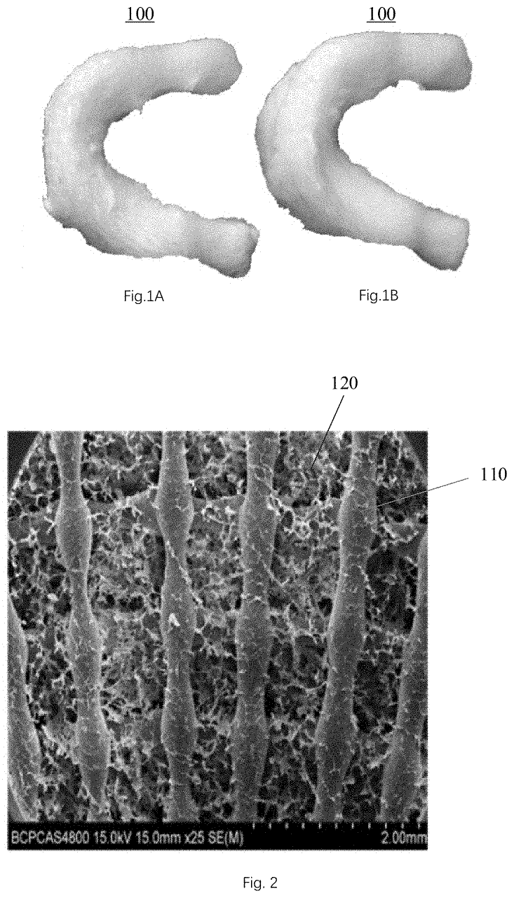

First, the tissue engineering meniscal composite scaffold provided in the first aspect of the embodiments of the present application will be described. FIG. 1A to FIG. 1B are schematic drawings of the tissue engineering meniscal composite scaffold 100 of an embodiment of the present application. FIG. 2 shows the scanning electron microscope cross section graph of the tissue engineering meniscal composite scaffold 100 of an embodiment of the present application. Turning now to FIGS. 1A, 1B and 2, the meniscal composite scaffold 100 of an embodiment of the present application includes a scaffold 110 and matrix material 120 composited within the scaffold 110.

The scaffold 110 is made by biocompatible and biodegradable polymer, so that it degrades as the new meniscus forms.

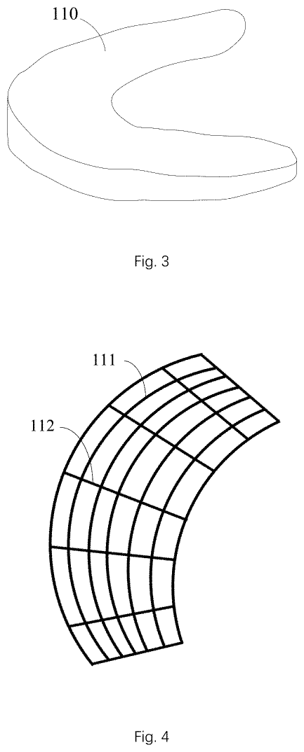

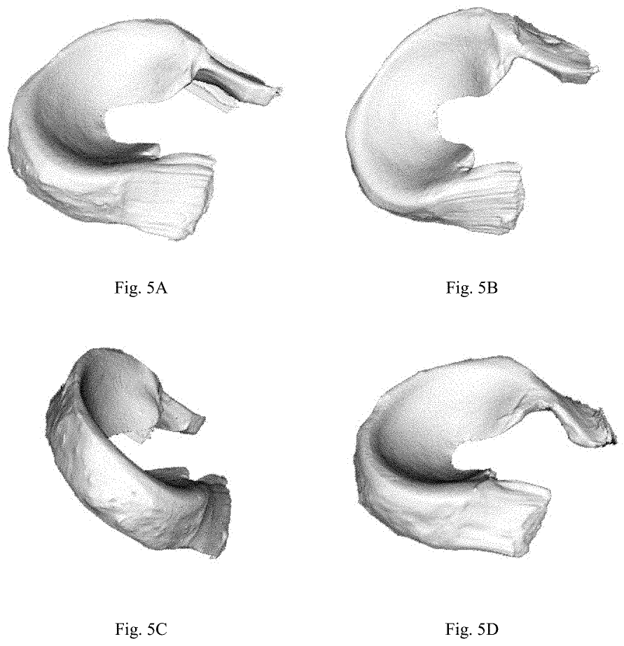

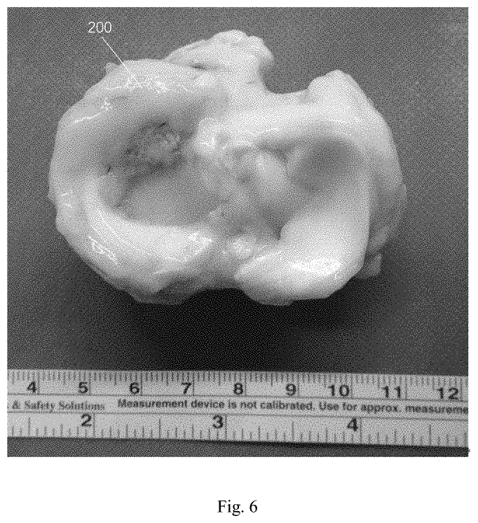

Turning now to FIG. 3. FIG. 3 is a schematic drawing of the scaffold of the tissue engineering meniscal composite scaffold of an embodiment of the present application. The scaffold is C-shaped and has a shape which is consistent with the original shape of the meniscus to be regenerated. In this embodiment, the original shape of the meniscus to be regenerated means the shape of the meniscus to be regenerated before it is damaged. It can be understood that said “consistent with” means the same or that medically acceptable deviation is allowed.

Turning now to FIG. 4. FIG. 4 is a schematic drawing showing the intersected first degradable polymer fibers and second degradable polymer fibers in the scaffold of an embodiment of the present application. The scaffold 110 comprises a plurality of first degradable polymer fibers 111 and a plurality of second degradable polymer fibers 112. The first degradable polymer fibers 111 is arc-shaped and extends along the radial direction of the scaffold 110, the plurality of first degradable polymer fibers 111 are arranged in parallel and spaced apart; the second degradable polymer fibers 112 can be linear shaped and extends along the circumferential direction of the scaffold, the plurality of second biodegradable polymer fibers 112 are radially arranged and spaced apart; the first biodegradable polymer fibers 111 intersects with the second biodegradable polymer fibers 112. The first degradable polymer fibers 111 form a multilayer intersection with the second degradable polymer fibers 112 and thereby generating a frame structure having a plurality of first apertures.

The scaffold 110, formed by biodegradable polymer fibers arranged according to a predetermined pattern, allows the meniscal composite scaffold 100 to have a good tensile elastic modulus and compression elastic modulus, and the desired mechanical properties. In particular, the scaffold 110 bio-mimic the structure characteristics of the collagen fibers in the meniscus to be regenerated, so that the newly formed meniscus has excellent shape, structure, mechanical properties and physiological functions.

In other embodiments, the scaffold 110 may comprise on its surface a plurality of third degradable polymer fibers which are radially arranged and spaced apart, so that the surface morphology of the meniscal composite scaffold 100 is more consistent with the surface morphology of the original meniscus. The inner portion of the scaffold 110 comprises a plurality of first degradable polymer fibers 111 and a plurality of second degradable polymer fibers 112; wherein the first degradable polymer fibers 111 is arc-shaped and extends along the radial direction of the scaffold 110, the plurality of first degradable polymer fibers 111 are arranged in parallel and spaced apart; the second degradable polymer fibers 112 can be linear shaped and extends along the circumferential direction of the scaffold, the plurality of second biodegradable polymer fibers 112 are radially arranged and spaced apart; the plurality of first biodegradable polymer fibers 111 form a multilayer intersection with the plurality of second biodegradable polymer fibers 112. In these embodiments, the scaffold 110 has a plurality of first apertures.

Further, the diameter of the first apertures of the scaffold 110 is 750 μm-1500 μm.

The matrix material 120 is composited inside the plurality of first apertures of the scaffold 110.

The meniscal composite scaffold 100 of the embodiments of the present application has a plurality of second apertures, the diameter of the second apertures is preferably 90 μm-150 μm.

The tissue engineering meniscal composite scaffold 100 of the embodiments of the present application has a configuration which fits an individual precisely, and is also well balanced between good mechanical properties and good biocompatibility, when implanted into the damaged area of the meniscus, the damaged meniscus could maintain normal joint activity and strength.

Meniscal cells, chondrocytes, mesenchymal stem cells, and the like are seeded into the plurality of second apertures of the meniscal composite scaffold 100, and since the tissue engineering meniscal composite scaffold 100 of the present application is able to provide for good microenvironment required for cell growth and facilitates growth, proliferation and re-differentiation of the cells under both in vivo and in vitro conditions, the regeneration the damaged meniscus in the avascular inner portion is promoted, and so that the newly formed meniscus has good shape, structure, mechanical properties and physiological function to protect the knee joint.

The diameter of the first degradable polymer fiber 111 is preferably 100 μm-300 μm.

The diameter of the second degradable polymer fiber 112 is preferably 100 μm-300 μm.

The diameter of the third degradable polymer fiber is preferably 100 μm-300 μm. Preferably, the porosity of the scaffold 110 is 85%-99%.

Preferably, the porosity of the meniscal composite scaffold 100 is 80%-95%.

Preferably, the tensile elastic modulus of the meniscal composite scaffold 100 is 10 MPa-100 MPa, and the compressive elastic modulus is 10 MPa-60 MPa.

The degradable polymer can be any polymer material that meets the biocompatibility and the mechanical properties requirements, such as one or more of polycaprolactone PCL, polyurethane PU, polylactic acid PLA, polylactic acid-glycolic acid copolymer PLGA, polylactic acid-polycaprolactone copolymer PCLA, polyamino acid PAA, and polyglycolic acid PGA.

Preferably, the average molecular weight of the degradable polymer is from 10,000 to 1,000,000.

The matrix material 120 may be a material that facilitates the attachment of the seed cells and active substances such as biological signal molecules, and is beneficial to cell growth, proliferation, and re-differentiation, the matrix material is preferably a natural material, such as one or more of a decellularized meniscus extracellular matrix, a decellularized chondrocyte extracellular matrix, a decellularized umbilical Wharton's jelly extracellular matrix, type I collagen, type II collagen, bacterial cellulose, silk protein and glycosaminoglycan.

The meniscal cells, chondrocytes and mesenchymal stem cells are seeded to the meniscal composite scaffold 100 and cultured for 50 h-350 h, such as 72 h-336 h, for example 150h-300 h, before used for the repair of partial or full damage of meniscus.

Next, the method for preparing a tissue engineering meniscal composite scaffold provided in the second aspect of the present application is described, the tissue engineering meniscal composite scaffold provided in the first aspect of the present application can be obtained by this method.

In an embodiment of the present application, the method for preparing a tissue engineering meniscal composite scaffold comprises the following steps:

a modeling step S100 to generate a three-dimensional data model of the undamaged meniscus to be regenerated.

As an example, step S100 includes the following steps.

Modeling step S110, obtaining the medical image data of the intact meniscus corresponding to the meniscus to be regenerated by the micro computed tomography (Micro-CT) or magnetic resonance imaging (MRI).

Said intact meniscus corresponding to the meniscus to be regenerated can be a meniscus of the intact knee joint of the patient corresponding to the meniscus to be regenerated.

A meniscal composite scaffold fitting precisely to an individual can be prepared according to the precise medical image data of the meniscus of the individual.

For example, see FIG. 5A to FIG. 5D showing the medical image data of a sheep medial meniscus obtained by Micro-CT imaging, and the three-dimensional data model of sheep medial meniscus is generated.

Step S120, the three-dimensional data model of the intact meniscus is generated using the medical image data of the intact meniscus corresponding to the meniscus to be regenerated by an image-processing software, and then the three-dimensional data model of the intact meniscus is mirror-imaged to obtain the three-dimensional data model of the meniscus to be regenerated before damage.

Step S130, the three-dimensional data model of the meniscus to be regenerated before damage is sliced to obtain the two-dimensional image data of the meniscus to be regenerated before damage.

Before step S130, the three-dimensional data model of the meniscus to be regenerated before damage can be subjected to local structural modifications and morphological optimization.

Printing step S200, using degradable polymer as raw material, a scaffold is printed according to the two-dimensional image data obtained from the three-dimensional data model of the meniscus to be regenerated before damage.

The degradable polymer may be a degradable polymer as described above.

Preferably, in step S200, the diameter of the print head is 100 μm-300 μm, the extrusion speed is 0.01 mm/s-0.03 mm/s, the printing speed is 5 mm/s-10 mm/s, and the layer thickness is 0.03 mm-0.10 mm.

Hydrophilic processing step S300, in which the scaffold is subjected to hydrophilic treatment.

In step S300, the scaffold may be subjected to hydrophilic treatment by using an alkaline etching treatment or a plasma treatment.

As an example of the hydrophilic treatment of the scaffold by the alkaline etching treatment, step S300 includes the following steps.

Step S310, the scaffold is washed several times by sterile tri-distilled water, for example 2 times, 3 times, or 4 times.

Step S320, the scaffold is soaked in an alkali solution to improve surface hydrophilicity.

The alkali solution may be an aqueous solution containing an alkali compound, such as sodium hydroxide, potassium hydroxide and the like. For example, the alkali solution is a 2 mol/L-6 mol/L sodium hydroxide aqueous solution, such as a 3 mol/L-5 mol/L sodium hydroxide aqueous solution.

The time of the soaking treatment may be 30 min-3 h, for example 1 h-2 h.

Step S330, the scaffold is washed with sterile tri-distilled water until it is neutral.

As an example of the hydrophilic treatment of the scaffold by the plasma treatment, an oxygen plasma can be used to treat the scaffold so that hydrophilic group such as hydroxyl group is formed on the surface of the scaffold to improve the hydrophilicity of the surface of the scaffold. Also, carbon dioxide plasma can be used to treat the scaffold, or a composite gas of oxygen and carbon dioxide can be used to perform plasma treatment on the scaffold to form hydrophilic groups such as hydroxyl groups, carbonyl groups, and carboxyl groups on the surface of the scaffold, this can improve the hydrophilicity of the surface of the scaffold.

A preparation step S400 for preparing lyophilized meniscal composite scaffold, in which a solution comprising matrix material is filled into the plurality of first apertures of the scaffold and the scaffold is subjected to lyophilization to obtain the lyophilized meniscal composite scaffold.

In the solution comprising matrix material, the matrix material may be a matrix material as described above, and the solvent may be water, ethanol, or the like. Preferably, in the solution comprising matrix material, the ratio between the mass of the matrix material and the volume of the solution is 1%-5%.

In step S400, a solution comprising matrix material can be filled into the plurality of first apertures of the scaffold by a method known in the art, for example, the solution comprising matrix material is injected into the first apertures of the scaffold through a syringe, or the scaffold is dipped into the solution comprising matrix material, so that the first apertures are fully soaked by the solution comprising matrix material.

In step S400, the scaffold filled with the solution comprising matrix material can be subjected to lyophilization by a method known in the art, for example, by using a vacuum freeze-dryer at −10° C.˜−60° C. and freeze dried for 12 h-48 h, such as 20 h-36 h, for example 24h-30 h. By lyophilization, the solvent is removed, and the homogeneous distribution the matrix material in the scaffold is achieved without changing the physical properties of the scaffold and the matrix material, so that the meniscal composite scaffold can have an excellent microenvironment.

Post-processing step S500, in which the lyophilized meniscal composite scaffold is subjected to cross-linking treatment and sterilization treatment to obtain a meniscal composite scaffold.

In step S500, the lyophilized meniscal composite scaffold can be subjected to cross-linking treatment and sterilization treatment by methods known in the art. For example, step S500 includes the following steps.

Step S510, the lyophilized meniscal composite scaffold is subjected to cross-linking treatment by one or more of a chemical process, an irradiation process and a heat dry process to obtain an initial meniscal composite scaffold.

The crosslinking treatment can improve the mechanical properties of the meniscal composite scaffold. The crosslinking of the matrix material can also improve the degradation rate of the matrix material to prevent shrinkage and maintain the morphology outside and the microstructure inside of the composite scaffold and thereby facilitating cell growth, proliferation and re-differentiation.

The lyophilized meniscal composite scaffold can be subjected to cross-linking treatment by a chemical process. For example, the lyophilized meniscal composite scaffold is added to a solution containing crosslinker to perform cross-linking treatment. The crosslinker may be one or more of carbodiimide (EDAC), N-hydroxy succinic acid imide (NHS), Genipin and glutaraldehyde (GDA), and the solvent may be one or more of water and ethanol.

The lyophilized meniscal composite scaffold can be subjected to cross-linking treatment by an irradiation process. For example, the lyophilized meniscal composite scaffold is subjected to cross-linking treatment by electron beam irradiation, UV irradiation or y-ray irradiation without the use of crosslinker to improve the biocompatibility of the meniscal composite scaffold. Also, in other embodiments, crosslinker can may be used simultaneously.

The lyophilized meniscal composite scaffold can be subjected to cross-linking treatment by a heat dry process.

Step S520, the initial meniscal composite scaffold is subjected to one or both of irradiation sterilization and ethylene oxide sterilization, to obtain meniscal composite scaffold.

As an example, the initial meniscal composite scaffold is subjected to irradiation sterilization using cobalt 60 irradiation. The initial meniscal composite scaffold can also be placed in ethylene oxide for sterilization.

The tissue engineering meniscal composite scaffold of the embodiments of the present application can be obtained using the method for preparing a tissue engineering meniscal composite scaffold of the embodiments of the present application, the tissue engineering meniscal composite scaffold has a configuration which fits an individual precisely, and is also well balanced between good mechanical properties and good biocompatibility, when implanted into the damaged area of the meniscus, the damaged meniscus could maintain normal joint activity and strength. The tissue engineering meniscal composite scaffold can also provide good microenvironment required for cell growth and facilitates growth, proliferation and re-differentiation of the cells under both in vivo and in vitro conditions, the regeneration the damaged meniscus in the avascular inner portion is greatly promoted, and so that the newly formed meniscus has good shape, structure, mechanical properties and physiological function.

### EXAMPLES

The following Examples describe the content disclosed in the present application in more detail. These Examples are only for illustrative purpose. It is obvious to those skilled in the art that various modifications and changes can be made within the scope of the disclosure of the present application. Unless otherwise stated, all parts, percentages, and ratios reported in the following Examples are based on weight, and all reagents used in the Examples are commercially available or synthesized by conventional methods, and can be used directly without further processing. The instruments used in the Examples are commercially available.

### Example 1

Medical image data of sheep medial meniscus was acquired by Micro-CT imaging, and three-dimensional reconstruction process was conducted to obtain the three-dimensional data model of sheep medial meniscus.

The decellularized meniscus extracellular matrix was prepared by a physical decellularization method, and an aqueous solution containing the decellularized meniscus extracellular matrix was prepared, wherein the ratio between the mass of the decellularized meniscus extracellular matrix and the volume of the aqueous solution was 2%.

Using polycaprolactone as raw material, the scaffold was printed according to the three-dimensional data model, which was C-shaped and had a shape which was consistent with the original shape of the meniscus to be regenerated, and comprised a plurality of first degradable polymer fibers extending along the circumferential direction of the scaffold and a plurality of second degradable polymer fibers extending along the radial direction of the scaffold; the first degradable polymer fibers formed a multilayer intersection with the second degradable polymer fibers and thereby generating a frame structure having a plurality of first apertures, the diameter of the first apertures was 750 μm-1500 μm.

The scaffold was subjected to oxygen plasma treatment, to improve the hydrophilicity of the scaffold.

The aqueous solution containing the decellularized meniscus extracellular matrix was filled into the plurality of first apertures in the scaffold subjected to hydrophilic treatment, the tissue engineering meniscal composite scaffold was obtained by lyophilization, chemical cross-linking process, and ethylene oxide sterilization. The meniscal composite scaffold had a plurality of second apertures, and the diameter of the second apertures was 90 μm-150 μm.

### Example 2

Same as Example 1 expect that the degradable polymer was polylactic acid-glycolic acid copolymer, and the matrix material was type I collagen, and the cross-linking was done by an irradiation process.

### Example 3

Same as Example 1 expect that the degradable polymer was polyurethane, the matrix material was bacterial cellulose, and the cross-linking was done by an irradiation process and irradiation sterilization using cobalt 60 irradiation was done.

### Example 4

Same as Example 1 expect that the scaffold was subjected to hydrophilic treatment by using an alkaline etching treatment, which includes: washing the scaffold 3 times with sterile tri-distilled water; immersing the scaffold in a 5 mol/L sodium hydroxide solution for 2 h; washing the scaffold with sterile tri-distilled water until the pH is neutral.

### Example 5

Same as Example 1 expect that the matrix material is silk protein, the cross-linking was done by an irradiation process and irradiation sterilization using cobalt 60 irradiation was done.

Tests

(1) Tensile test: the obtained tissue engineering meniscal composite scaffold was clipped to obtain a rectangular sample with 10 mm width, 20 mm length, and 5 mm thickness, the sample was soaked in physiological saline for 3 h, and was hold by the gripper of a mechanical testing machine, the sample was stretched at a tensile rate of 5 mm/min until broken to obtain the tensile stress and tensile strain curves, and the tensile elastic modulus was calculated according to the stress-strain curve. The test results of Examples 1 to 5 are shown in Table 1 below.

TABLE 1

| | Exam- | Exam- | Exam- | Exam- | Exam- |
|---|---|---|---|---|---|
| | ple 1 | ple 2 | ple 3 | ple 4 | ple 5 |
| tensile elastic | 32.3 | 31.6 | 31.1 | 32.0 | 30.5 |
| modulus/MPa | | | | | |

(2) Compression test: the obtained tissue engineering meniscal composite scaffold was clipped to obtain a rectangular sample with 5 mm width, 5 mm length, and 5 mm thickness, the sample was soaked in physiological saline for 3 h, and then was placed between the two pressure plates of a mechanical testing machine, the sample was compressed at a compression rate of 5 mm/min to 30% strain to obtain compressive stress and compressive strain curves. The compressive elastic modulus was calculated based on the stress-strain curve. The test results of Examples 1 to 5 are shown in Table 2 below.

TABLE 2

| | Exam- | Exam- | Exam- | Exam- | Exam- |
|---|---|---|---|---|---|
| | ple 1 | ple 2 | ple 3 | ple 4 | ple 5 |
| compressive | 18.9 | 18.5 | 19.0 | 18.8 | 17.8 |
| elastic modulus/MPa | | | | | |

(3) test of the repair of sheep medial meniscus damage: the knee joint was opened after the sheep was anesthetized, and the medial meniscus was removed, and the obtained tissue engineering meniscal composite scaffold was sutured in situ to the damaged meniscus, and then the muscle, fascia, and skin, were sewed up and the knee joint was closed. The knee joint was opened again 3 months after the operation for examination.

No infection or ulceration was observed in the implantation site of the sheep using the meniscal composite scaffold of Examples 1 to 5. It can be seen that the meniscal composite scaffolds of the Examples of the present application have good biocompatibility. The meniscal composite scaffolds of Examples 1 to 5 were partially degraded, and there were still residues; new meniscal tissue was formed at the site where the meniscal composite scaffold was degraded. Sampling and analysis revealed that the shape of the new meniscal tissue was consistent with the original intact meniscus, and new collagen fibers arranged in parallel and intersected; the new meniscal tissue was tested to have full physiological functions of a meniscus and provided an effective protection to knee cartilage. Among them, the meniscal composite scaffold of Example 4 reproduced the original meniscus better in terms of shape, structure, mechanical properties and physiological functions, and the effects were also better.

In conclusion, the tissue engineering meniscal composite scaffolds of Example 1 to Example 5 of the present application have porous structure, and their mechanical strength is suitable for meniscal transplantation, and especially suitable for the repair of the damaged meniscus in the avascular inner portion and protecting the knee joint.

The above are only some particular embodiments of the present application, and the scope of the present application is not limited thereto, any skilled in the art can readily conceive various equivalent modifications or changes within the scope of the present application, and these modifications or changes are within the protection scope of this application. Therefore, the protection scope of this application depends on the claims.

1. A tissue engineering meniscal composite scaffold including:
a scaffold, which is C-shaped and has a shape which is consistent with the original shape of the meniscus to be regenerated, the scaffold comprises a plurality of first degradable polymer fibers extending along the circumferential direction of the scaffold and a plurality of second degradable polymer fibers extending along the radial direction of the scaffold; the first degradable polymer fibers form a multilayer intersection with the second degradable polymer fibers and thereby generating a frame structure having a plurality of first apertures, the diameter of the first apertures is 750 μm-1500 μm;
matrix material, which is composited inside the plurality of first apertures to form a meniscal composite scaffold having a plurality of second apertures, the diameter of the second apertures is 90 μm-150 μm.

2. The tissue engineering meniscal composite scaffold according to claim 1, wherein the diameter of one or both of the first degradable polymer fibers and the second degradable polymer fibers is 100 μm-300 μm.

3. The tissue engineering meniscal composite scaffold according to claim 1, wherein the porosity of the scaffold is 85%-99%; and the porosity of the meniscal composite scaffold is 80%-95%.

4. The tissue engineering meniscal composite scaffold according to claim 1, wherein the first degradable polymer fibers and the second degradable polymer fibers are made from one or more of the degradable polymer selected from the group consisting of polycaprolactone PCL, polyurethane PU, polylactic acid PLA, polylactic acid-glycolic acid copolymer PLGA, polylactic acid-polycaprolactone copolymer PCLA, polyamino acid PAA, and polyglycolic acid PGA.

5. The tissue engineering meniscal composite scaffold according to claim 4, wherein the average molecular weight of the degradable polymer is from 10,000 to 1,000,000.

6. The tissue engineering meniscal composite scaffold according to claim 1, wherein the matrix material is one or more material selected from the group consisting of decellularized meniscus extracellular matrix, decellularized chondrocyte extracellular matrix, decellularized umbilical Wharton's jelly extracellular matrix, type I collagen, type II collagen, bacterial cellulose, silk protein and glycosaminoglycan.

7. The tissue engineering meniscal composite scaffold according to claim 1, wherein the tensile elastic modulus of the meniscal composite scaffold is 10 MPa-100 MPa, and the compressive elastic modulus is 10 MPa-60 MPa.

8. A method for preparing a tissue engineering meniscal composite scaffold comprising:
a modeling step to generate a three-dimensional data model of the meniscus to be regenerated before damage;
a printing step, in which a scaffold is printed according to the three-dimensional data model using degradable polymer as raw material, and the scaffold is C-shaped and has a shape which is consistent with the original shape of the meniscus to be regenerated, the scaffold comprises a plurality of first degradable polymer fibers extending along the circumferential direction of the scaffold and a plurality of second degradable polymer fibers extending along the radial direction of the scaffold; the first degradable polymer fibers form a multilayer intersection with the second degradable polymer fibers and thereby generating a frame structure having a plurality of first apertures, the diameter of the first apertures is 750 μm-1500 μm;
a hydrophilic treatment step, in which the scaffold is subjected to hydrophilic treatment;
a preparation step for preparing lyophilized meniscal composite scaffold, in which a solution comprising matrix material is filled into the plurality of first apertures of the scaffold and the scaffold is subjected to lyophilization to obtain the lyophilized meniscal composite scaffold;
a post-processing step, in which the lyophilized meniscal composite scaffold is subjected to cross-linking treatment and sterilization treatment to obtain a meniscal composite scaffold having a plurality of second apertures, the diameter of the second apertures is 90 μm-150 μm.

9. The method according to claim 8, wherein in the printing step, the diameter of the print head is 100 μm-300 μm, the extrusion speed is 0.01 mm/s-0.03 mm/s, the printing speed is 5 mm/s-10 mm/s, and the layer thickness is 0.03 mm-0.10 mm.

10. The method according to claim 8, wherein in the hydrophilic treatment step, the scaffold is subjected to hydrophilic treatment by using an alkaline etching treatment or a plasma treatment.

11. The method according to claim 8, wherein in the preparation step for preparing lyophilized meniscal composite scaffold, the ratio between the mass of the matrix material and the volume of the solution in the solution comprising matrix material is 1%-5%.

12. The method according to claim 8, wherein the post-processing step includes:
the lyophilized meniscal composite scaffold is subjected to cross-linking treatment by one or more of a chemical process, an irradiation process and a heat dry process to obtain an initial meniscal composite scaffold,
the initial meniscal composite scaffold is subjected to one or both of irradiation sterilization and ethylene oxide sterilization, to obtain the meniscal composite scaffold.
